SEQUENCE LISTING (3 sequences)

SEQ ID NO: 1 - RNA, 54 nt, unidentified
  source 1..54: mol_type = mRNA; note = TP53 sequence
tcttttcctatcctgagtagtggtaatctactgggacggaacagctttgaggtg

SEQ ID NO: 2 - protein, 7 aa, unidentified
  source 1..7: note = TP53 sequence
GRNSFEV

SEQ ID NO: 3 - protein, 18 aa, unidentified
  source 1..18: note = TP53 sequence
SFPILSSGNLLGRNSFEV

(19) United States
(12) Patent Application Publication
(10) Pub. No.: US 2024/0374575 A1
(43) Pub. Date: Nov. 14, 2024

(54) METHODS AND COMPOSITIONS FOR TREATING CANCER

(71) Applicant: President and Fellows of Harvard College, Cambridge, MA (US)

(72) Inventors: Joan Brugge, Cambridge, MA (US); Matthew D. Shair, Cambridge, MA (US)

(21) Appl. No.: 18/648,806

(22) Filed: Apr. 29, 2024

Related U.S. Application Data
(60) Provisional application No. 63/462,716, filed on Apr. 28, 2023.

(51) Int. Cl. A61K 31/437; A61K 31/282; A61K 31/4355; A61K 31/436; A61K 31/4375; A61K 31/4985; A61K 31/5025; A61K 31/519; A61K 31/53; A61K 45/06

(52) U.S. Cl. CPC A61K 31/437; A61K 31/282; A61K 31/4355; A61K 31/436; A61K 31/4375; A61K 31/4985; A61K 31/5025; A61K 31/519; A61K 31/53; A61K 45/06

(57) ABSTRACT

Provided herein are methods of treating or preventing cancer in a subject, the method comprising administering to the subject a compound having the structure of Formula I, Formula II, or Formula III, or a pharmaceutically acceptable salt thereof.

### CROSS-REFERENCE TO RELATED APPLICATIONS

This application claims the benefit of the U.S. Provisional Application Ser. No. 63/462,716 filed Apr. 28, 2023, the entire contents of which is incorporated herein by reference.

### BACKGROUND

There remains a critical need to develop novel therapies for combating cancer.

### SUMMARY

Provided herein are methods of treating or preventing cancer in a subject, the method comprising administering to the subject a compound having the structure of Formula I, Formula II, or Formula III, or a pharmaceutically acceptable salt thereof. Also provided herein are methods of decreasing the activity of or lowering the levels of cancer associated fibroblasts in a subject afflicted with cancer, the method comprising administering to the subject a compound having the structure of Formula I, Formula II, or Formula III, or a pharmaceutically acceptable salt thereof.

In some aspects, provided herein are methods of decreasing the activity of or lowering the levels of NNMT in a subject afflicted with cancer, the method comprising administering to the subject a compound having the structure of Formula I, Formula II, or Formula III, or a pharmaceutically acceptable salt thereof.

In some embodiments, the cancer is ovarian cancer. In other embodiments, the cancer is pancreatic cancer. In some embodiments, the compound is administered with an additional cancer agent. The addition cancer agent may be a chemotherapy (e.g., oxaliplatin) or an immune checkpoint inhibitor. The immune checkpoint inhibitor may comprises an inhibitor of an immune checkpoint protein selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof.

In some embodiments, the compound and additional cancer agent exhibit synergy when administered conjointly.

The disclosure also relates to methods of treating or preventing cancer in a subject and methods of inhibiting tumor growth in a subject.

In certain embodiments, the present application discloses methods of using compounds of Formula I:

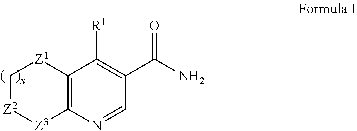

or a pharmaceutically acceptable salt thereof, wherein R¹, Z¹, Z², Z³ and x are as defined herein.

In certain embodiments, the present application discloses methods of using compounds of Formula II:

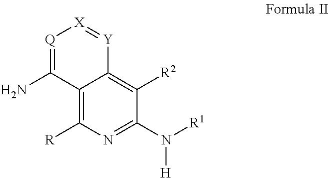

or a pharmaceutically acceptable salt thereof, wherein R, R¹, R², Q, X, and Y are as defined herein.

In certain embodiments, the present application discloses methods of using compounds of Formula III:

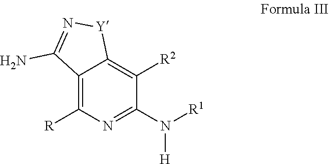

or a pharmaceutically acceptable salt thereof, wherein R, R¹, R², and Y′ are as defined herein.

### BRIEF DESCRIPTION OF THE DRAWINGS

FIG. 1 shows NNMT is the major enzyme of NAM clearance.

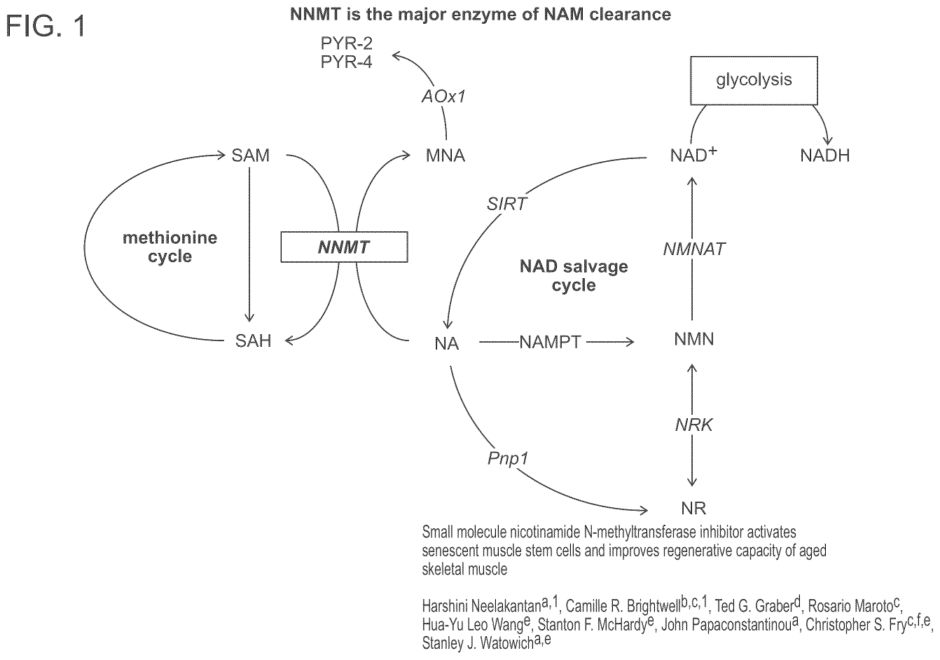

FIG. 2 shows that NNMT as a regulator of ovarian cancer fibroblasts.

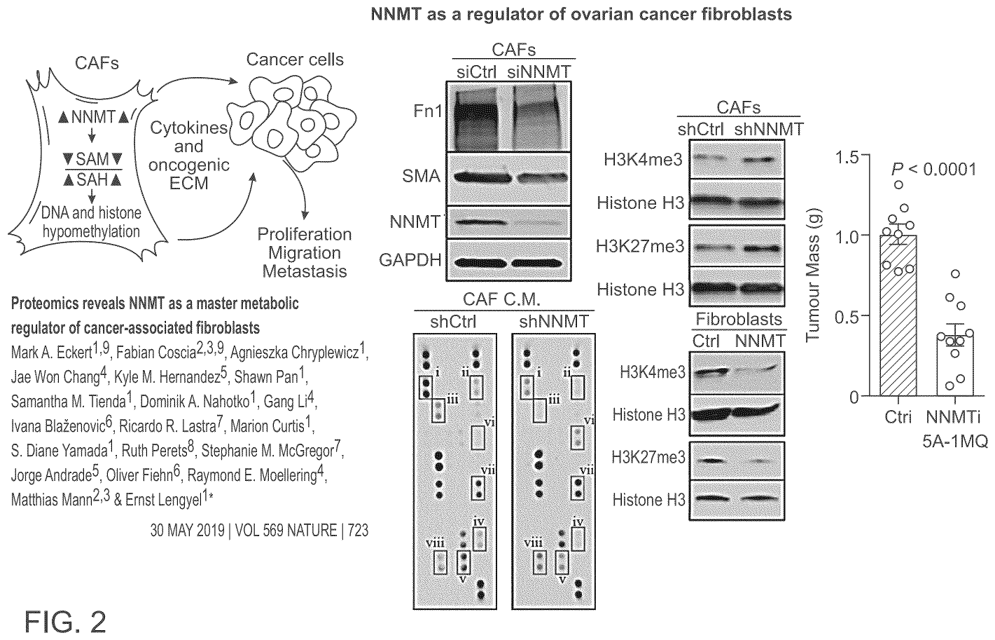

FIG. 3 shows ovarian CAF cultures.

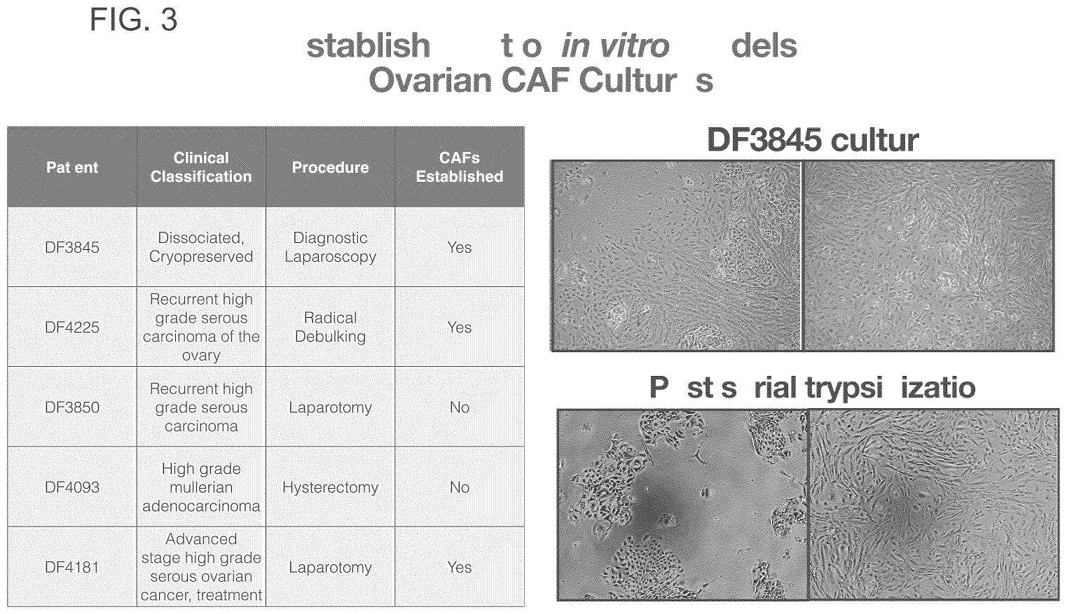

FIG. 4 shows confirmation of fibroblast origins of CAF models.

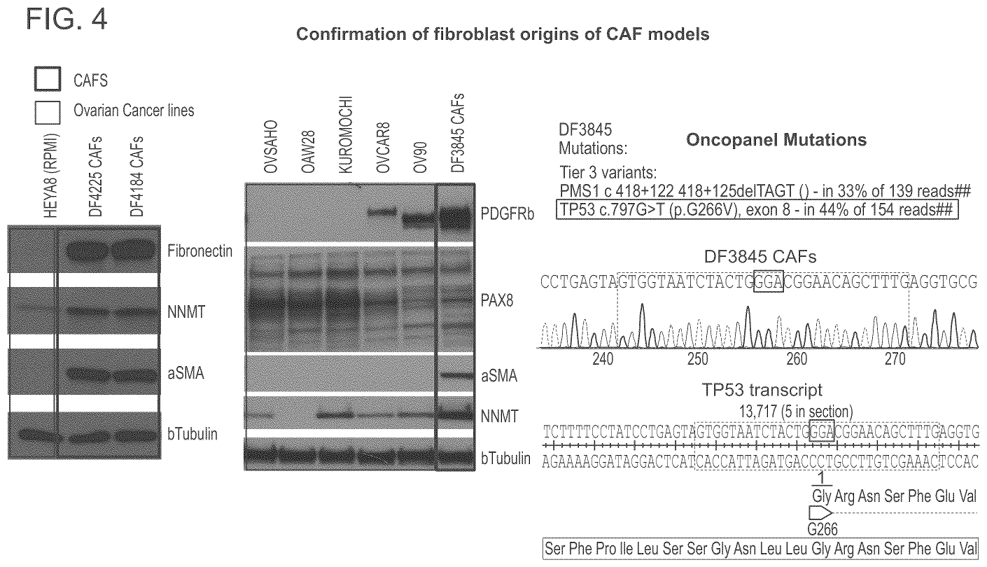

FIG. 5 shows NNMT inhibitor treatment in CAFs.

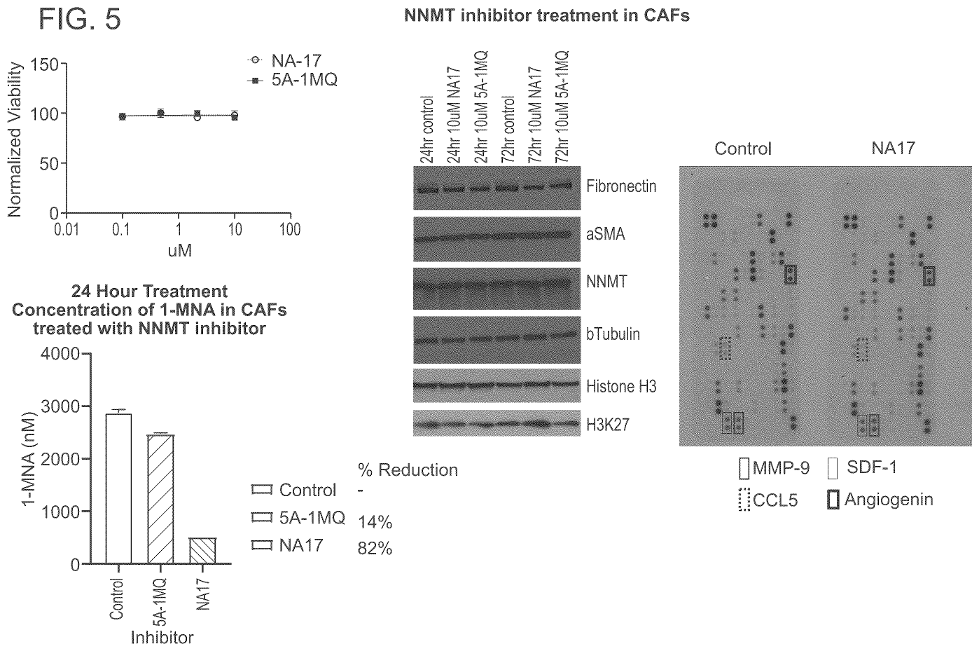

FIG. 6 shows NNMT inhibitor treatment in CAFs.

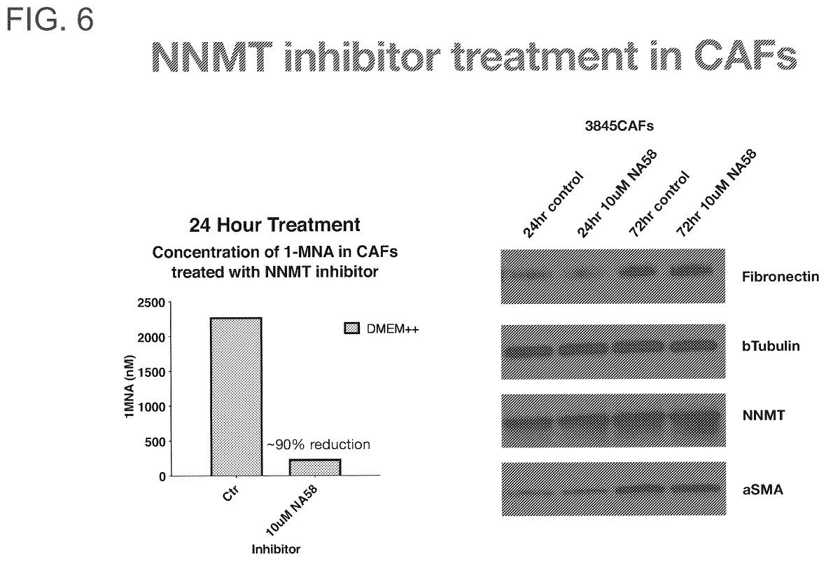

FIG. 7 shows in vivo models.

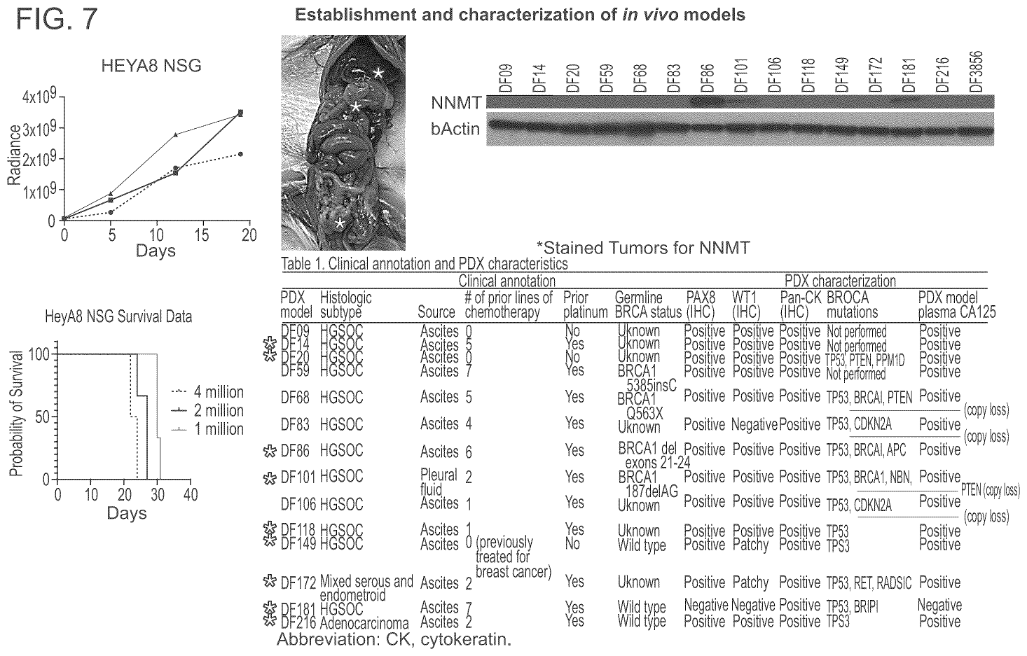

FIG. 8 shows staining in primary and xenograft tumors.

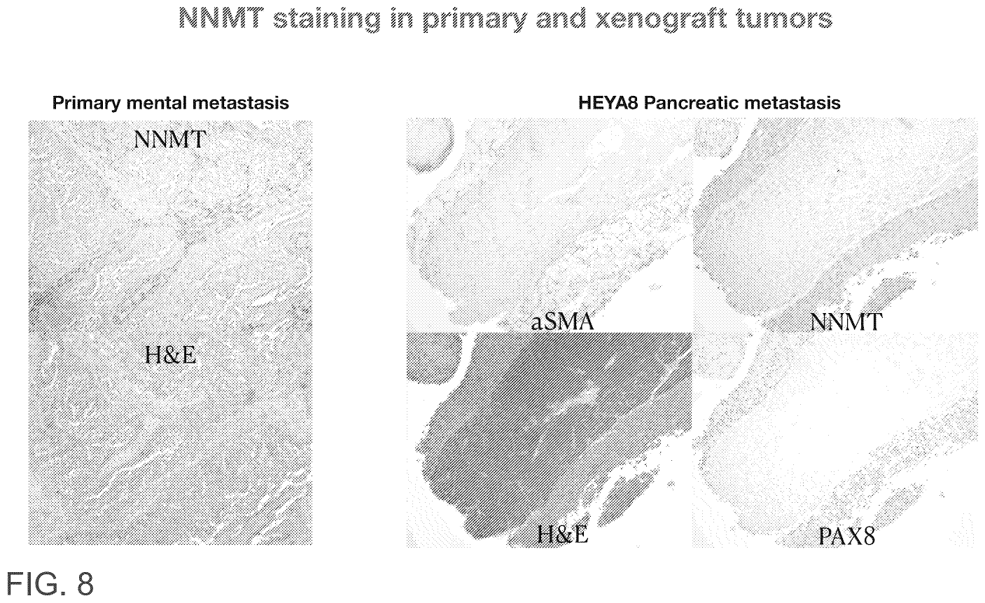

FIG. 9 shows NNMT staining in primary and xenograft tumors.

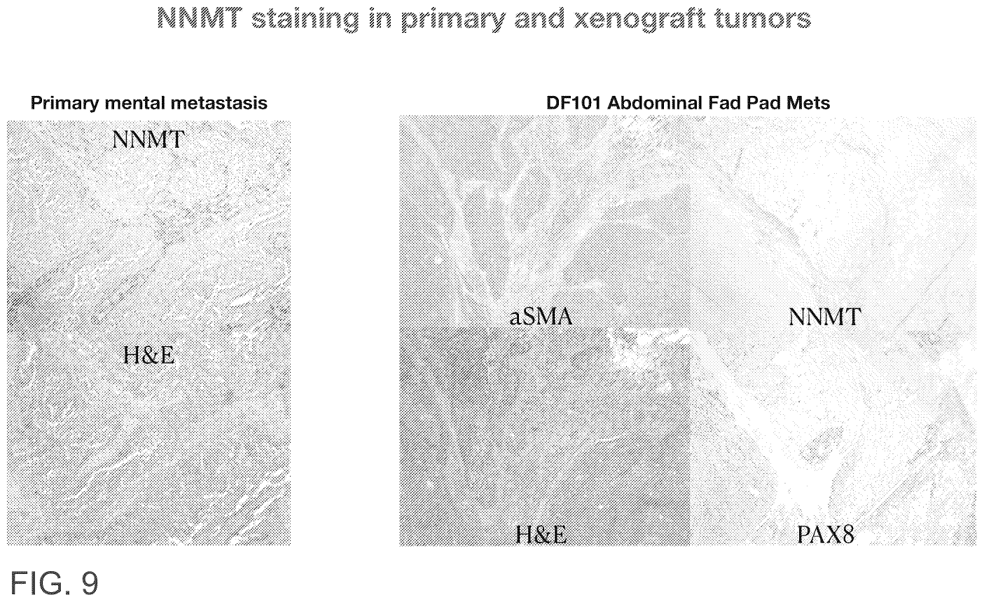

FIG. 10 shows generation of HEYA8 NNMT KO models.

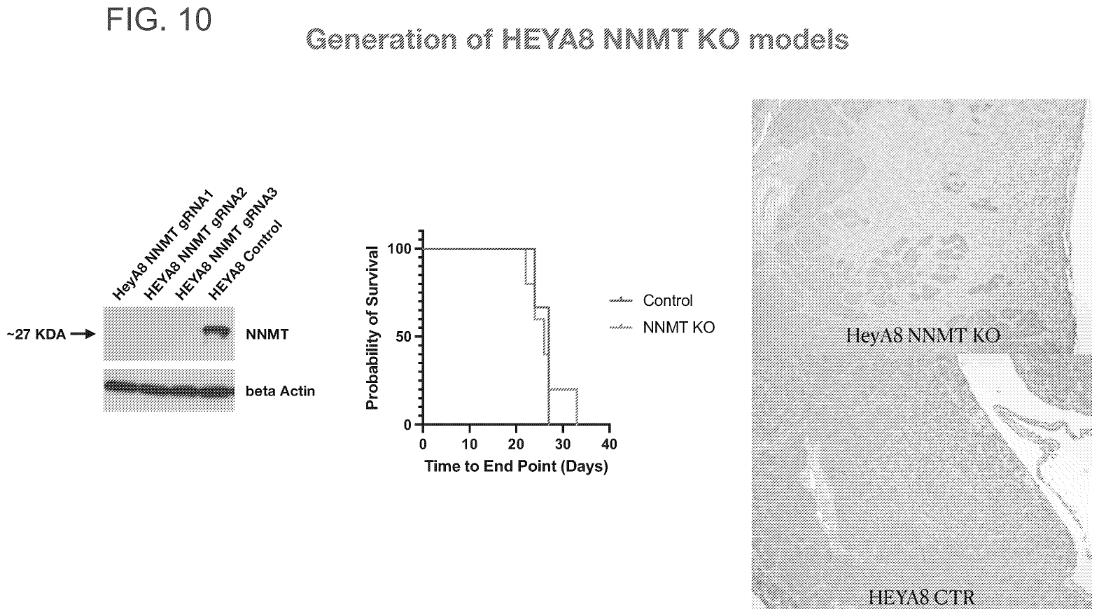

FIG. 11 shows NNMT study plan (HeyA8).

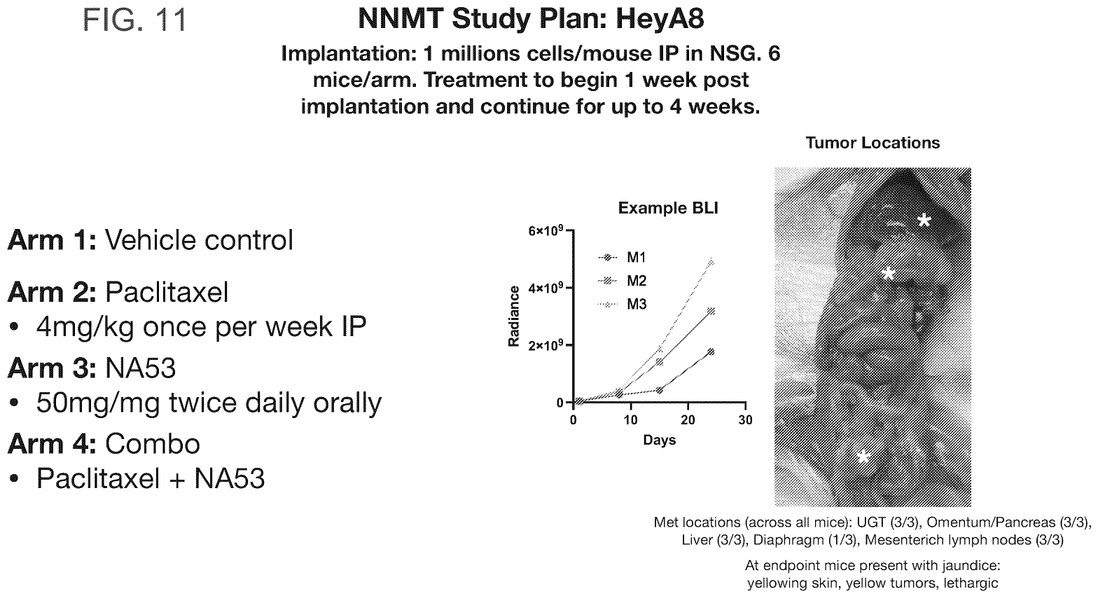

FIG. 12 shows NNMT study plan (CT26).

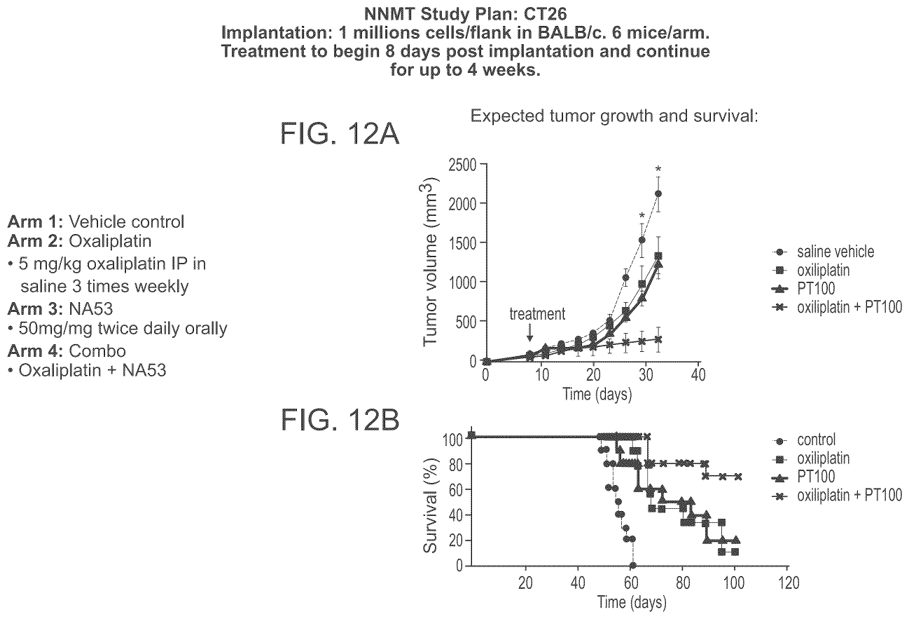

FIG. 13 shows CT26 study: NA53 50 mpk BID.

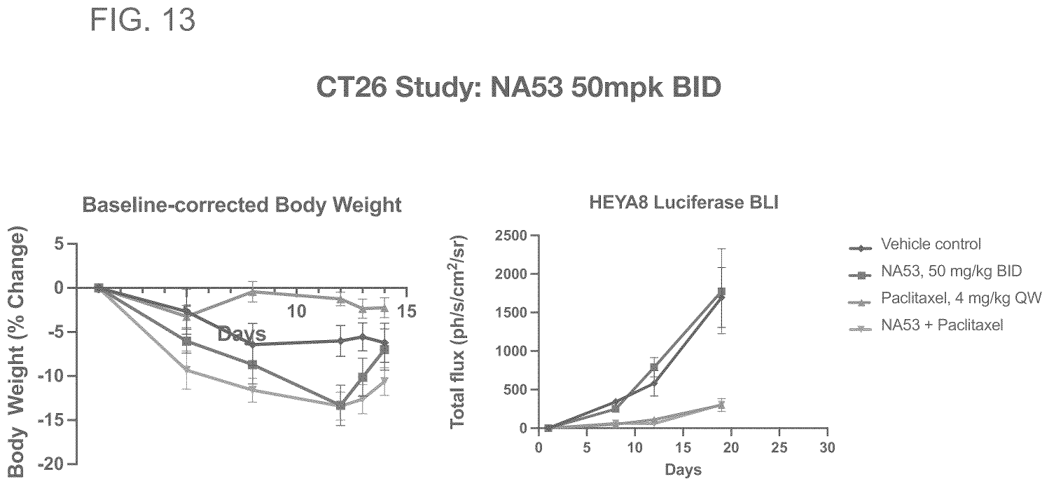

FIG. 14 shows CT26: NA53 50mpk BID.

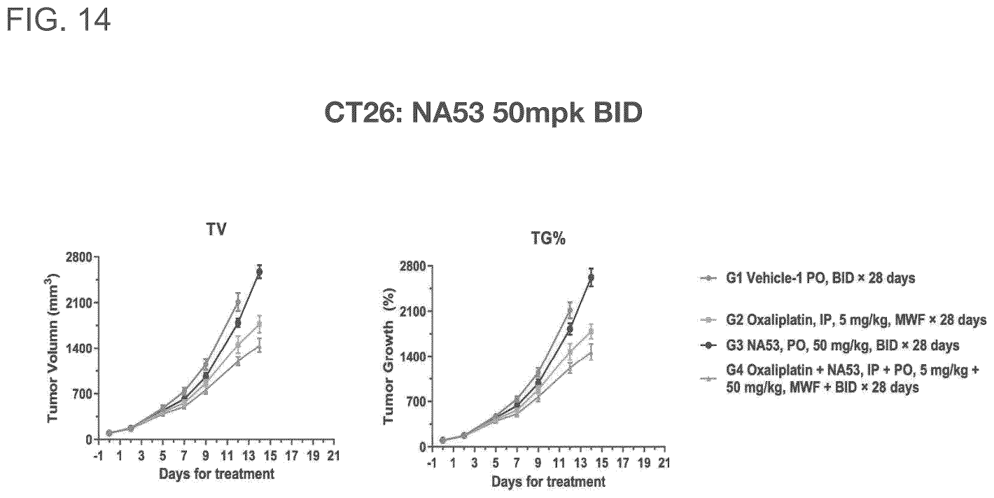

### DETAILED DESCRIPTION

Provided herein are methods of treating or preventing cancer in a subject, the method comprising administering to the subject a compound having the structure of Formula I, Formula II, or Formula III or a pharmaceutically acceptable salt thereof. Also provided herein are methods of decreasing the activity of or lowering the levels of cancer associated fibroblasts in a subject afflicted with cancer, the method comprising administering to the subject a compound having the structure of Formula I, Formula II, or Formula III, or a pharmaceutically acceptable salt thereof.

In some aspects, provided herein are methods of decreasing the activity of or lowering the levels of NNMT in a subject afflicted with cancer, the method comprising administering to the subject a compound having the structure of Formula I, Formula II, or Formula III, or a pharmaceutically acceptable salt thereof.

The disclosure also relates to methods of treating or preventing cancer in a subject and methods of inhibiting tumor growth in subject. Tumors are heterogeneous, comprising cancer cells and an elaborate microenvironment. Cancer-associated fibroblasts (CAFs) are myofibroblasts (fibroblasts with smooth muscle cell characteristics) present in the tumor microenvironment that support the tumor through paracrine signaling and the production of an extracellular matrix. CAFs play crucial roles in almost all aspects of tumor biology including survival, resistance, metastasis and immune cell evasion. CAFs have now been identified in solid tumors of almost all tissues types, sometimes outnumbering any other cell type in a tumor and associated with a poor prognosis in patients. CAF-driven build-up of extracellular matrix has been shown to prevent the infiltration of effector immune cells and activated T cells. Thus, reducing the presence of CAFs in tumors may improve responses and resistance to immunotherapies. Given their intimate role in cancer maintenance, progression and resistance to targeted therapies and immunotherapies, therapeutics specifically targeting CAFs hold enormous promise as a new approach in cancer treatment. However, few targets that are specific to CAFs (versus normal fibroblasts) have been identified.

To identify targets that are specific to CAFs, proteins that are differentially expressed in human CAFs but not tumor cells or normal stroma have been identified. Specifically, biopsy samples from patients with high-grade serous carcinoma metastases (HGSC—the most common form of ovarian cancer) underwent laser microdissection to separate tumor cells from stroma followed by mass spectrometry. It was found that expression of nicotinamide N-methyltransferase (NNMT) was increased in stroma of HGSC metastases compared to tumor cells or normal stroma. NNMT was also highly expressed in breast and colon cancer stroma. Importantly, NNMT was required to maintain the CAF phenotype. Furthermore, tumor burden in animal models was reduced when NNMT was knocked down or inhibited with a small molecule inhibitor. These studies indicate that NNMT is a CAF-selective therapeutic target and its inhibition with small molecules reverses the CAF phenotype and reduces tumor burden.

The subject may have a cancer may be any cancer that expresses NNMT. The cancer may be any cancer that has high levels or expression of NNMT. As used herein, “high levels or expression of NNMT” can refer to protein or nucleic acid (e.g., mRNA) levels of NNMT. In addition, “high levels or expression” may be determined by comparison to cancer-free cells of the same or different type. “High levels or expression” may be determined by comparison to any other cells or tissue in the tumor microenvironment (including tumor cells) or outside the tumor microenvironment that are predicted to express average levels of NNMT or are not predicted to express NNMT.

The cancer may be any cancer that exhibits high levels of CAFs. As used herein, “high levels of CAFs” may refer to numerical levels of CAFs or the levels of a biomarker associated with CAFs. In addition, “high levels” may be determined by comparison to cancer-free tissue of the same or different type. “High levels or expression” may be determined by comparison to any other tissue in the tumor microenvironment or outside the tumor microenvironment that is predicted to express average levels of CAFs or are not predicted to express CAFs. The cancer may be a refractory cancer (e.g., refractory to an additional cancer agent described herein).

Provided herein are screening methods of determining whether a subject would be a candidate for a therapy described herein by first determining if the cancer or tumor in the subject comprises high levels or expression of NNMT and/or high level of CAFs. If the subject high levels or expression of NNMT and/or high levels of CAFs, the method may further comprise administering a compound or pharmaceutical composition disclosed herein.

### Definitions

The articles “a” and “an” are used herein to refer to one or to more than one (i.e. to at least one) of the grammatical object of the article. By way of example, “an element” means one element or more than one element.

As used herein, the term “acyl” is art-recognized and refers to a group represented by the general formula hydrocarbylC(O)—, preferably alkylC(O)—.

As used herein, the term “alkoxy” refers to an alkyl group, preferably a lower alkyl group, having an oxygen attached thereto. Representative alkoxy groups include methoxy, —OCF₃, ethoxy, propoxy, tert-butoxy and the like.

As used herein, the term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

As used herein, the term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

As used herein, the term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

As used herein, the term “amide”, as used herein, refers to a group

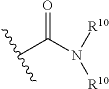

wherein each R¹⁰ independently represent a hydrogen or hydrocarbyl group, or two R¹⁰ are taken together with the N atom to which they are attached complete a heterocycle having from 4 to 8 atoms in the ring structure.

As used herein, the terms “amine” and “amino” are art-recognized and refer to both unsubstituted and substituted amines and salts thereof, e.g., a moiety that can be represented by

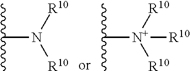

wherein each R¹⁰ independently represents a hydrogen or a hydrocarbyl group, or two R¹⁰ are taken together with the N atom to which they are attached complete a heterocycle having from 4 to 8 atoms in the ring structure.

As used herein, the term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

As used herein, the terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 3-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

As used herein, a “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

As used herein, the terms “halo” and “halogen” as used herein means halogen and includes chloro, fluoro, bromo, and iodo.

As used herein, the terms “hetaralkyl” and “heteroaralkyl”, as used herein, refers to an alkyl group substituted with a hetaryl group.

As used herein, the term “heteroalkyl”, as used herein, refers to a saturated or unsaturated chain of carbon atoms and at least one heteroatom, wherein no two heteroatoms are adjacent.

As used herein, the terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

As used herein, the term “heteroatom” as used herein means an atom of any element other than carbon or hydrogen. Preferred heteroatoms are nitrogen, oxygen, and sulfur.

As used herein, the terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can also be substituted by oxo groups. For example, “heterocyclyl” encompasses both pyrrolidine and pyrrolidinone.

As used herein, the term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

As used herein, the term “hydroxyalkyl”, as used herein, refers to an alkyl group substituted with a hydroxy group.

As used herein, the term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “oxo” refers to a carbonyl group. When an oxo substituent occurs on an otherwise saturated group, such as with an oxo-substituted cycloalkyl group (e.g., 3-oxo-cyclobutyl), the substituted group is still intended to be a saturated group. When a group is referred to as being substituted by an “oxo” group, this can mean that a carbonyl moiety (i.e., —C(═O)—) replaces a methylene unit (i.e., —CH₂—).

As used herein, the terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the term “administering” means the actual physical introduction of a composition into or onto (as appropriate) a subject. Any and all methods of introducing the composition into subject are contemplated according to the invention; the method is not dependent on any particular means of introduction and is not to be so construed. Means of introduction are well-known to those skilled in the art, and also are exemplified herein.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting, or transitory, that can be associated with the administration of the pharmaceutical composition.

As used herein, the term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds in a formulation represented above can be replaced with the corresponding suitable prodrug, e.g., wherein a hydroxyl in the parent compound is presented as an ester or a carbonate or carboxylic acid present in the parent compound is presented as an ester.

The terms “cancer” or “tumor” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., myelomas like multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), myeloma, multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma, or breast carcinoma. The epithelial cancers may be characterized in various other ways including, but not limited to, serous, endometrioid, mucinous, clear cell, Brenner, or undifferentiated. In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The terms “prevent,” “preventing,” “prevention,” and the like refer to reducing the probability of developing a disease, disorder, or condition in a subject, who does not have, but is at risk of or susceptible to developing a disease, disorder, or condition.

The “tumor microenvironment” is an art-recognized term and refers to the cellular environment in which the tumor exists, and includes, for example, interstitial fluids surrounding the tumor, surrounding blood vessels, immune cells, other cells, fibroblasts, signaling molecules, and the extracellular matrix.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Compounds

In certain aspects, the present application discloses methods of treating cancer by administering substituted multicyclic compounds and pharmaceutical compositions thereof to a subject. In particular, such compounds disclosed herein are useful as inhibitors of nicotinamide N-methyltransferase (NNMT).

NNMT catalyzes the methylation of nicotinamide using S-adenosylmethionine (SAM) as a cofactor, which generates 1-methyl nicotinamide (1-MNA). Not wishing to be bound by theory, high expression of NNMT may maintain the CAF phenotype by reducing SAM levels, which would lead to DNA and histone hypomethylation and epigenetic/transcriptional alterations that maintain the CAF cell state. This theory is supported by three observations: 1) NNMT is a ‘methyl sink’ that reduces SAM levels and histone methylation 2) NNMT knockdown in CAFs increases trimethylation of histone 3 lysines 4 and 27 and 3) inhibition of histone methyltransferase EZH2 rescues NNMT knockdown and restores the CAF phenotype (α-SMA and collagen contractility).

Thus, the compounds disclosed herein can be used as inhibitors of NNMT, which is particularly useful with respect to treating cancer, such as cancerous tumors associated with CAFs having an increased expression of NNMT.

### Formula 1

In certain embodiments, the present application discloses compounds of Formula I or a pharmaceutically acceptable salt thereof:

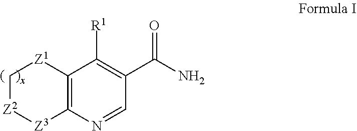

wherein
Z¹ is O, NR⁴ or CHR²;
Z² is O or CHR³;
Z³ is O or NH;
R¹ is H, halo, cyano, unsubstituted or substituted alkyl, unsubstituted or substituted cycloalkyl, or unsubstituted or substituted alkynyl;
R² and R³ are each, independently, H, cyano, unsubstituted or substituted alkyl, unsubstituted or substituted alkynyl, alkyl-OR⁵, or alkyl-SR⁶; or
if x is 0, then R² and R³, taken together with the carbon atoms to which they are attached, may form a fused 3-, 4-, 5- or 6-membered carbocyclic ring;
R⁴ is H, alkyl, or acyl;
R⁵ and R⁶ are each, independently, H, CHF₂, CF₃, unsubstituted or substituted alkyl, unsubstituted or substituted alkynyl, or unsubstituted or substituted cycloalkyl; and
x is an integer selected from 0 and 1;
provided that Z² and Z³ are not both O; and if x is 0, then Z¹ and Z² are not both O and Z¹ and Z³ are not both O.

In certain embodiments, the present application discloses compounds of Formula I or a pharmaceutically acceptable salt thereof:

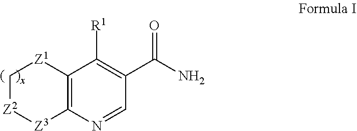

wherein
Z¹ is O, NR⁴ or CHR²;
Z² is O or CHR³;
Z³ is O or NH;
R¹ is H, halo, cyano, unsubstituted or substituted alkyl (e.g., fluoroalkyl), unsubstituted or substituted cycloalkyl, or unsubstituted or substituted alkynyl;
R² and R³ are each, independently, H, cyano, alkyl, or alkynyl; or
if x is 0, then R² and R³, taken together with the carbon atoms to which they are attached, may form a fused 3-, 4-, 5- or 6-membered carbocyclic ring;
R⁴ is H, alkyl, or acyl; and
x is an integer selected from 0 and 1;
provided that Z² and Z³ are not both O; and if x is 0, then Z¹ and Z² are not both O and Z¹ and Z³ are not both O.

In some embodiments, Z¹ and Z² are not both O.

In some embodiments, x is 0. In some embodiments, x is 1.

In some embodiments, Z¹ is CHR². In some embodiments, R² is H. In some embodiments, Z¹ is O. In some embodiments, Z¹ is NR⁴.

In some embodiments, Z² is CHR³. In some embodiments, R³ is H. In some embodiments, R³ is cyano. In some embodiments, R³ is alkyl, preferably lower alkyl, more preferably methyl. In some embodiments, R³ is alkynyl, preferably lower alkynyl, more preferably ethynyl. In some embodiments, Z² is O.

In some embodiments, R² and R³, taken together with the carbon atoms to which they are attached, form a fused 3-membered carbocyclic ring.

In some embodiments, Z³ is NH. In some embodiments, Z³ is O.

In some embodiments, R¹ is alkyl, preferably lower alkyl, more preferably methyl. In some embodiments, R¹ is halo, preferably chloro. In some embodiments, R¹ is substituted alkyl, preferably substituted lower alkyl, more preferably fluorine-substituted lower alkyl.

In some embodiments, R⁴ is is alkyl, preferably lower alkyl, more preferably methyl.

In some embodiments, the compound of Formula I has the structure:

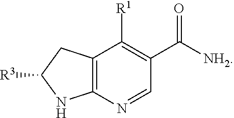

In some embodiments, R¹ is H or CH₃.

In some embodiments, R¹ is halo.

In some embodiments, R¹ is Cl.

In some embodiments, R³ is unsubstituted C₁₋C₆ alkyl.

In some embodiments, R³ is CH₃.

In some embodiments, R³ is unsubstituted C₂₋C₆ alkynyl. In other embodiments, R³ is substituted C₂₋C₆ alkynyl. In some embodiments, when substituted, the C₂₋C₆ alkynyl is substituted with CF₃.

In some embodiments, R³ is alkyl-OR⁵ or alkyl-SR⁶. In some embodiments, R⁵ and R⁶ are each, independently, CHF₂, CF₃, CH₃, CH(CH₃)₂, or CH(CH₂)₂.

In some embodiments, the compound of Formula I has the structure:

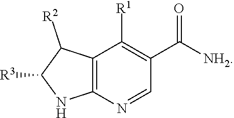

In some embodiments, the compound of Formula I has the structure:

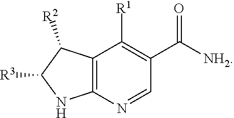

In some embodiments, the compound of Formula I has the structure:

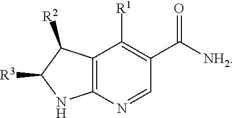

In some embodiments, the compound of Formula I has the structure:

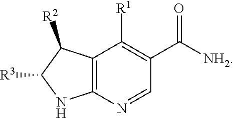

In some embodiments, R¹ is H or CH₃.

In some embodiments, R¹ is halo.

In some embodiments, R¹ is Cl.

In some embodiments, R² is unsubstituted C₁₋C₆ alkyl.

In some embodiments, R³ is unsubstituted C₁₋C₆ alkyl.

In some embodiments, R¹, R², and R³ are each CH₃.

Exemplary compounds of Formula I are depicted in Table 1.

TABLE 1
Exemplary Compounds of Formula I

| Compound No. | Structure |
|---|---|
| 1 | 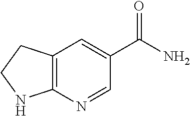 [SMILES: NC(=O)c1cnc2c(c1)CCN2] |
| 2 | 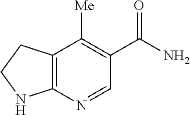 [SMILES: Cc1c(C(N)=O)cnc2c1CCN2] |
| 3 | 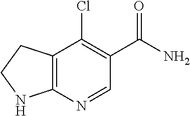 [SMILES: NC(=O)c1cnc2c(c1Cl)CCN2] |
| 4 | 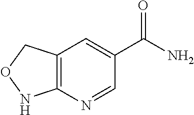 [SMILES: NC(=O)c1cnc2c(c1)CON2] |
| 5 | 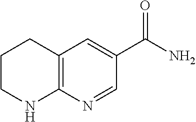 [SMILES: NC(=O)c1cnc2c(c1)CCCN2] |
| 6 | 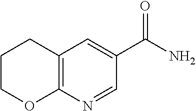 [SMILES: NC(=O)c1cnc2c(c1)CCCO2] |
| 7 | 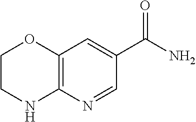 [SMILES: NC(=O)c1cnc2c(c1)OCCN2] |
| 8 | 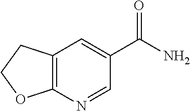 [SMILES: NC(=O)c1cnc2c(c1)CCO2] |
| 9 | 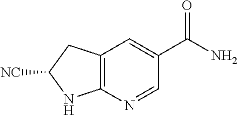 [SMILES: N#C[C@@H]1Cc2cc(C(N)=O)cnc2N1] |
| 10 | 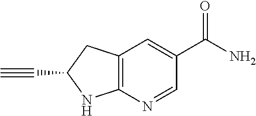 |
| 11 | 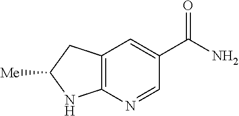 [SMILES: C[C@@H]1Cc2cc(C(N)=O)cnc2N1] |
| 12 | 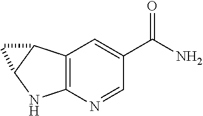 [SMILES: NC(=O)c1cnc2c(c1)[C@H]1C[C@H]1N2] |
| 13 | 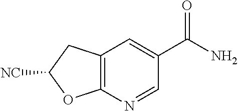 [SMILES: N#C[C@@H]1Cc2cc(C(N)=O)cnc2O1] |
| 14 | 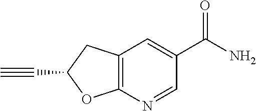 |
| 15 | 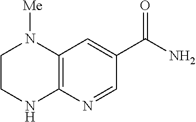 [SMILES: CN1CCNc2ncc(C(N)=O)cc21] |
| 16 | 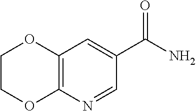 [SMILES: NC(=O)c1cnc2c(c1)OCCO2] |
| 17 | 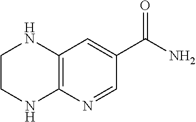 [SMILES: NC(=O)c1cnc2c(c1)NCCN2] |

Additional exemplary compounds of Formula I are depicted in Table 2.

TABLE 2
Exemplary Compounds of Formula I

| Compound No. | Structure |
|---|---|
| 18 | 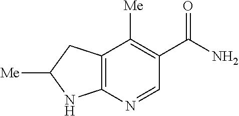 [SMILES: Cc1c(C(N)=O)cnc2c1CC(C)N2] |
| 19A | 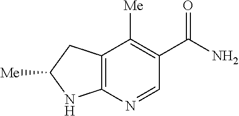 [SMILES: Cc1c(C(N)=O)cnc2c1C[C@@H](C)N2] |
| 19B | 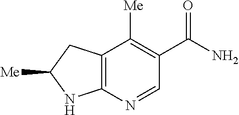 [SMILES: Cc1c(C(N)=O)cnc2c1C[C@H](C)N2] |
| 20A | 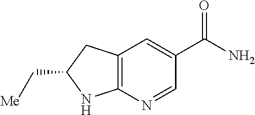 [SMILES: CC[C@@H]1Cc2cc(C(N)=O)cnc2N1] |
| 20B | 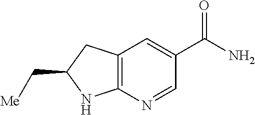 [SMILES: CC[C@H]1Cc2cc(C(N)=O)cnc2N1] |
| 21 | 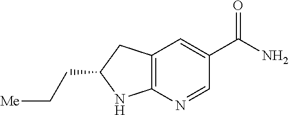 [SMILES: CCC[C@@H]1Cc2cc(C(N)=O)cnc2N1] |
| 22 | 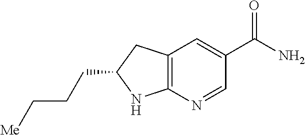 [SMILES: CCCC[C@@H]1Cc2cc(C(N)=O)cnc2N1] |
| 23 | 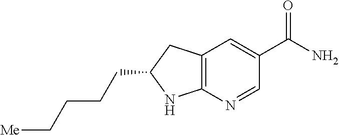 [SMILES: CCCCC[C@@H]1Cc2cc(C(N)=O)cnc2N1] |
| 24 | 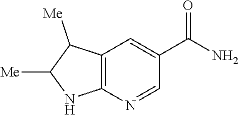 [SMILES: CC1Nc2ncc(C(N)=O)cc2C1C] |
| 25A | 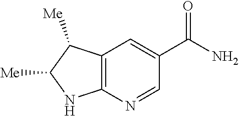 [SMILES: C[C@@H]1c2cc(C(N)=O)cnc2N[C@@H]1C] |
| 25B | 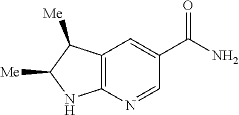 [SMILES: C[C@@H]1Nc2ncc(C(N)=O)cc2[C@@H]1C] |
| 26A | 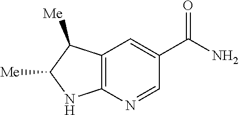 [SMILES: C[C@H]1Nc2ncc(C(N)=O)cc2[C@@H]1C] |
| 26B | 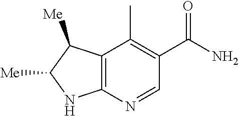 |
| 27 | 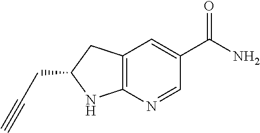 |
| 28 | 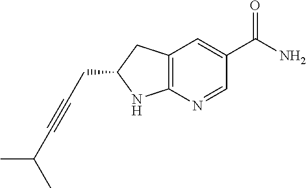 [SMILES: CC(C)C#CC[C@@H]1Cc2cc(C(N)=O)cnc2N1] |
| 29 | 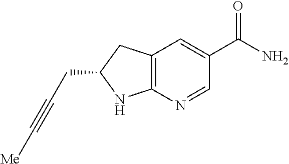 [SMILES: CC#CC[C@@H]1Cc2cc(C(N)=O)cnc2N1] |
| 30 | 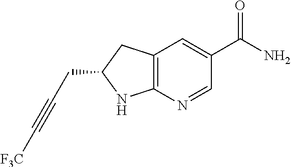 [SMILES: NC(=O)c1cnc2c(c1)C[C@@H](CC#CC(F)(F)F)N2] |
| 31 | 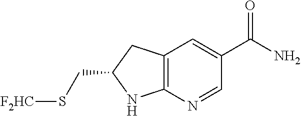 [SMILES: NC(=O)c1cnc2c(c1)C[C@@H](CSC(F)F)N2] |
| 32 | 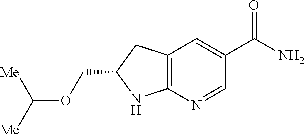 [SMILES: CC(C)OC[C@@H]1Cc2cc(C(N)=O)cnc2N1] |
| 33 | 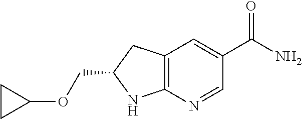 [SMILES: NC(=O)c1cnc2c(c1)C[C@@H](COC1CC1)N2] |
| 34 | 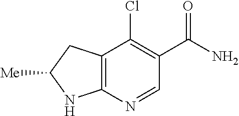 [SMILES: C[C@@H]1Cc2c(ncc(C(N)=O)c2Cl)N1] |

### Formula II and III

In certain embodiments, the present application discloses compounds of Formula II or a pharmaceutically acceptable salt thereof:

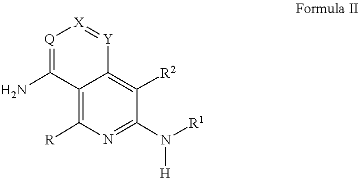

wherein
Q is N or CH;
X is N or CH;
Y is N of CH;
R is H or alkyl;
R¹ is alkyl;
R² is H; or
R¹ and R², taken together with the atoms to which they are attached, form an optionally-substituted fused 5-, 6-, or 7-membered heterocyclic ring,
provided that at least one of Q, X, and Y is N.

In certain embodiments, the compound has the structure:

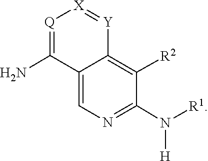

In certain embodiments, Q, X, and Y are not N, CH, and N, respectively.

In certain embodiments, R is H.

In certain embodiments, R is alkyl. In other embodiments, R is CH₃. In certain embodiments, R¹ is alkyl; and R² is H. In certain embodiments, Q is N.

In certain embodiments, X is N. In other embodiments, X is CH.

In certain embodiments, Y is N. In other embodiments, Y is CH.

In certain embodiments, the compound having the structure:

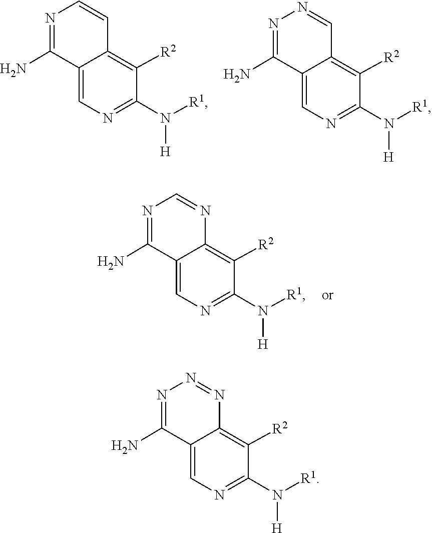

In certain embodiments, the compound having the structure:

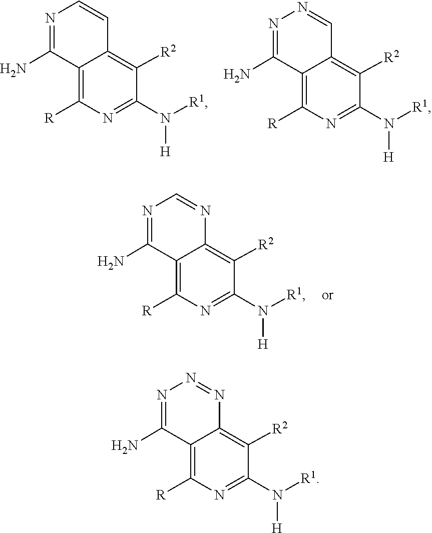

In certain embodiments, the compound having the structure:

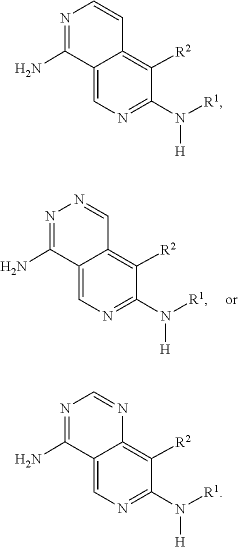

In certain embodiments, the compound having the structure:

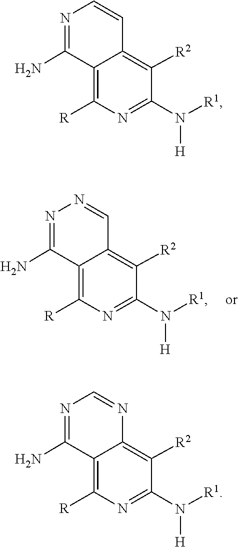

In certain embodiments, R is unsubstituted C₁₋C₆ alkyl.

In certain embodiments, R is CH₃.

In certain embodiments, R¹ is C₁-C₆ alkyl. In other embodiments, R¹ is C₁-C₄ alkyl. In other embodiments, R¹ is CH₃.

In certain embodiments, R¹ and R², taken together with the atoms to which they are attached, form an unsubstituted or substituted fused 5-membered heterocyclic ring.

In certain embodiments, the compound having the structure:

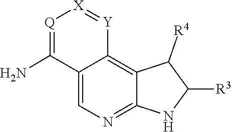

wherein
R³ and R⁴ are each, independently, H, cyano, unsubstituted or substituted alkyl, unsubstituted or substituted alkynyl, alkyl-OR⁵, or alkyl-SR⁶; and
R⁵ and R⁶ are each, independently, H, CHF₂, CF₃, unsubstituted or substituted alkyl, unsubstituted or substituted alkynyl, or unsubstituted or substituted cycloalkyl.

In certain embodiments, Q is N.

In certain embodiments, X is N. In other embodiments, X is CH.

In certain embodiments, Y is N. In other embodiments, Y is CH.

In certain embodiments, the compound having the structure:

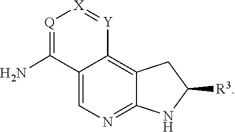

In certain embodiments, the compound having the structure:

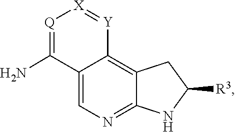

wherein Q, X, and Y are not N, CH, and N, respectively. In certain embodiments, the compound having the structure:

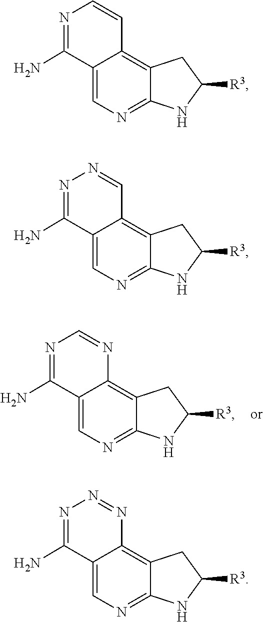

In certain embodiments, the compound having the structure:

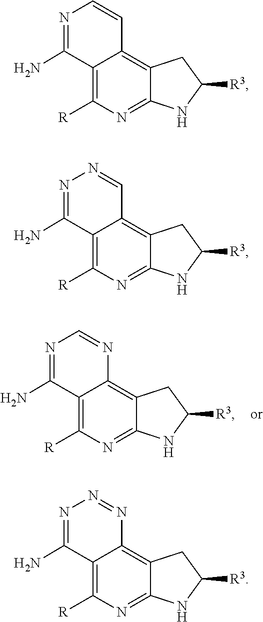

In certain embodiments, the compound having the structure:

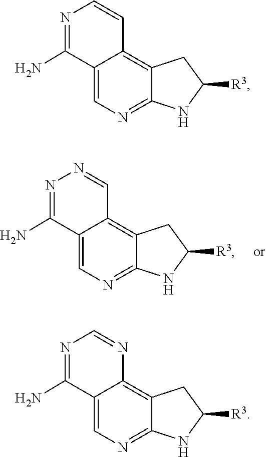

In certain embodiments, the compound having the structure:

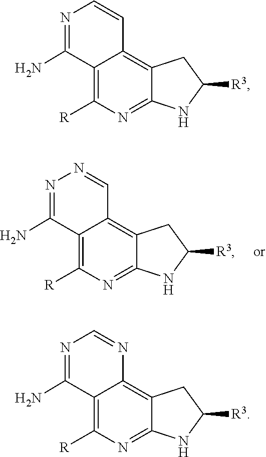

In certain embodiments, R is unsubstituted C₁₋C₆ alkyl.

In certain embodiments, R is CH₃.

In certain embodiments, R³ is unsubstituted C₁₋C₆ alkyl.

In certain embodiments, R³ is CH₃.

In certain embodiments, R³ is unsubstituted C₂₋C₆ alkynyl. In other embodiments, R³ is substituted C₂₋C₆ alkynyl.

In certain embodiments, C₂₋C₆ alkynyl is substituted with CF₃.

In certain embodiments, R³ is alkyl-OR⁵ or alkyl-SR⁶.

In certain embodiments, R⁵ and R⁶ are each, independently, CHF₂, CF₃, CH₃, CH(CH₃)₂, or CH(CH₂)₂.

In certain embodiments, the compound having the structure:

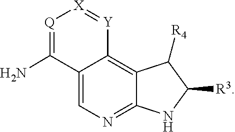

In certain embodiments, the compound having the structure:

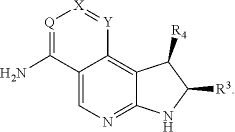

In other embodiments, the compound having the structure:

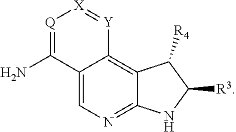

In certain embodiments, the compound having the structure:

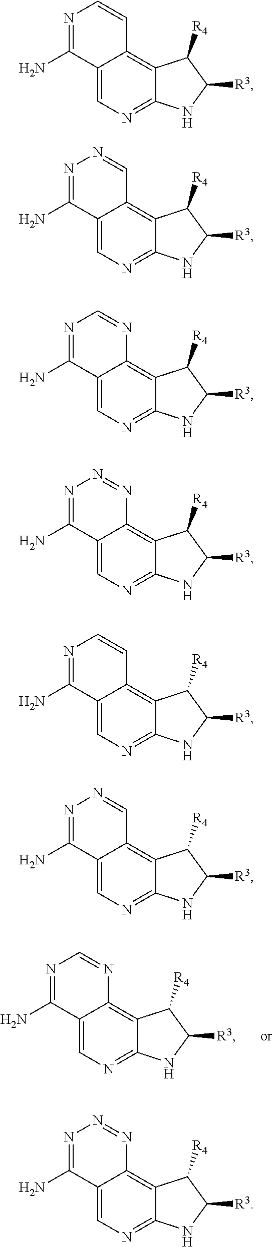

In certain embodiments, R³ and R⁴ are each unsubstituted C₁-C₆ alkyl.

In certain embodiments, R³ and R⁴ are each CH₃.

In certain embodiments, the compound having the structure:

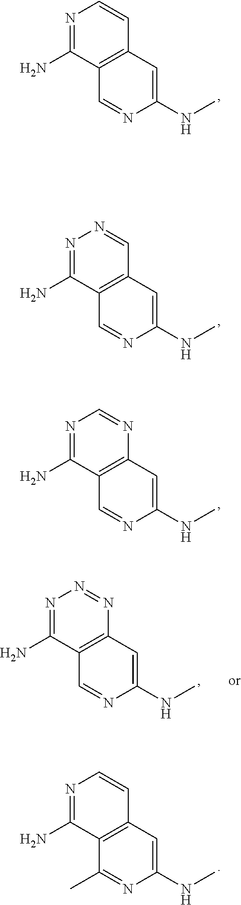

In certain embodiments, the compound having the structure:

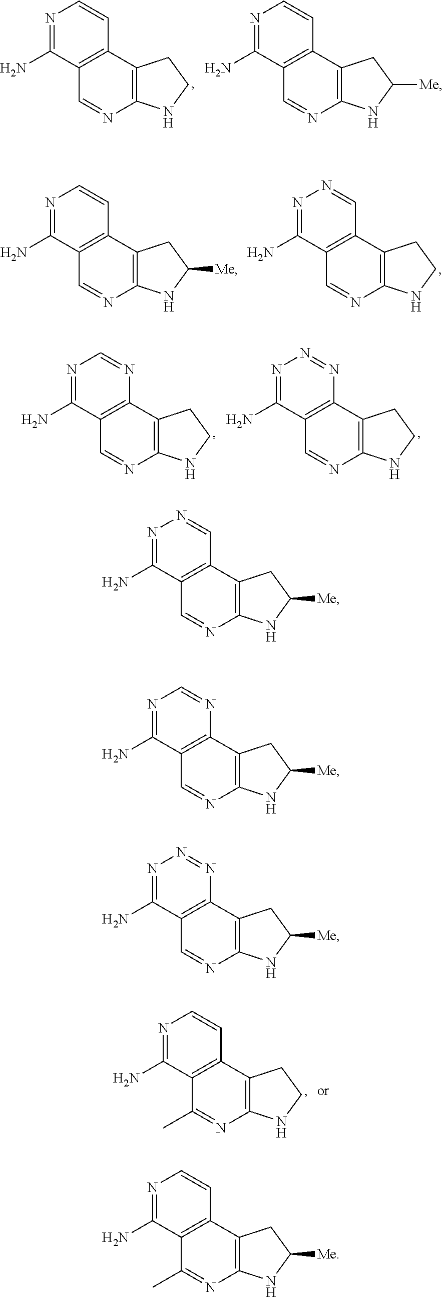

In certain embodiments, the compound having the structure:

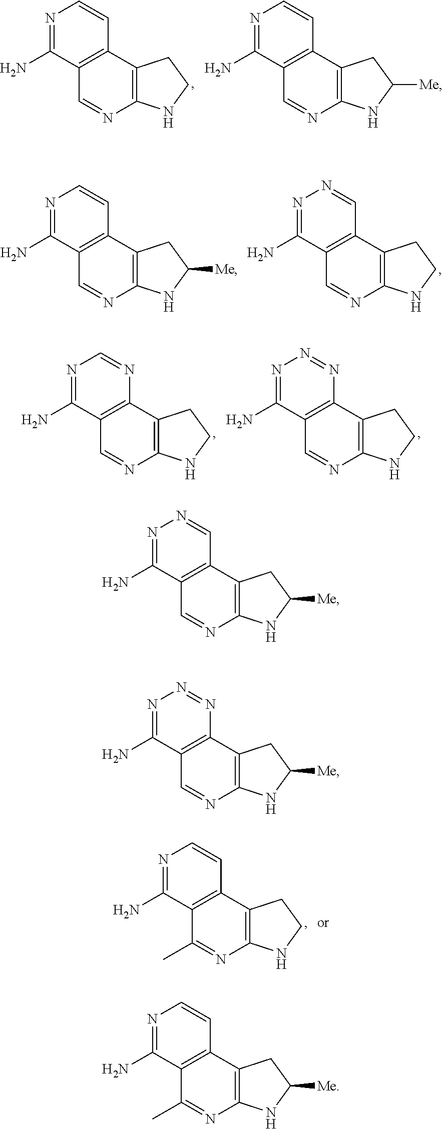

In certain embodiments, the compound having the structure:

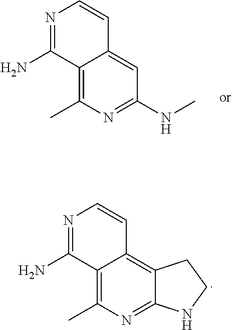

In certain embodiments, the present application discloses compounds of Formula III or a pharmaceutically acceptable salt thereof:

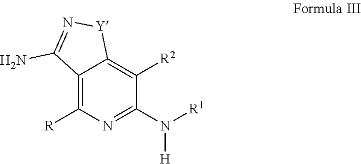

wherein
Y′ is S or O;
R is H or alkyl;
R¹ is alkyl;
R² is H; or
R¹ and R², taken together with the atoms to which they are attached, form an unsubstituted or substituted fused 5-, 6-, or 7-membered heterocyclic ring.

In certain embodiments, the compound has the structure:

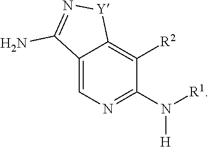

In certain embodiments, R is H.

In certain embodiments, R is alkyl. In other embodiments, R is CH₃.

In certain embodiments, R¹ is alkyl; and R² is H.

In certain embodiments, wherein Y is S. In other embodiments, wherein Y′ is O.

In certain embodiments, the compound having the structure:

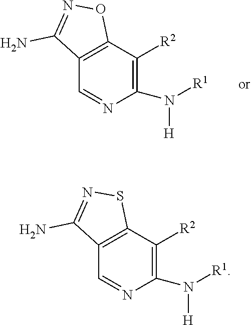

In certain embodiments, R¹ is C₁-C₆ alkyl. In other embodiments, R¹ is C₁-C₄ alkyl. In other embodiments, R¹ is CH₃.

In certain embodiments, R¹ and R², taken together with the atoms to which they are attached, form an unsubstituted or substituted fused 5-membered heterocyclic ring.

In certain embodiments, the compound having the structure:

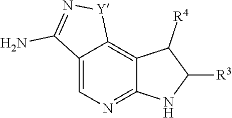

wherein
R³ and R⁴ are each, independently, H, cyano, unsubstituted or substituted alkyl, unsubstituted or substituted alkynyl, alkyl-OR⁵, or alkyl-SR⁶; and
R⁵ and R⁶ are each, independently, H, CHF₂, CF₃, unsubstituted or substituted alkyl, unsubstituted or substituted alkynyl, or unsubstituted or substituted cycloalkyl.

In certain embodiments, wherein Y′ is S. In other embodiments, Y′ is O.

In certain embodiments, the compound having the structure:

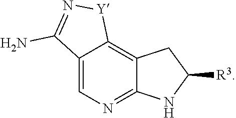

In certain embodiments, the compound having the structure:

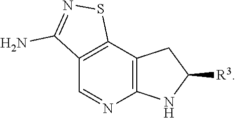

In certain embodiments, R³ is unsubstituted C₁₋C₆ alkyl.

In certain embodiments, R³ is CH₃.

In certain embodiments, R³ is unsubstituted C₂₋C₆ alkynyl. In other embodiments, R³ is substituted C₂₋C₆ alkynyl.

In certain embodiments, the C₂₋C₆ alkynyl is substituted with CF₃.

In certain embodiments, R³ is alkyl-OR⁵ or alkyl-SR⁶.

In certain embodiments, R⁵ and R⁶ are each, independently, CHF₂, CF₃, CH₃, CH(CH₃)₂, or CH(CH₂)₂.

In certain embodiments, the compound having the structure:

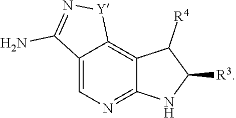

In certain embodiments, the compound having the structure:

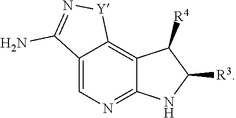

In certain embodiments, the compound having the structure:

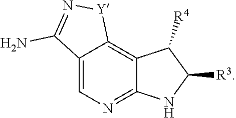

In certain embodiments, R³ and R⁴ are each unsubstituted C₁-C₆ alkyl. In other embodiments, R³ and R⁴ are each CH₃.

In certain embodiments, the compound having the structure:

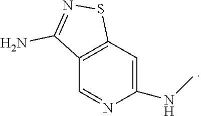

In certain embodiments, the compound having the structure:

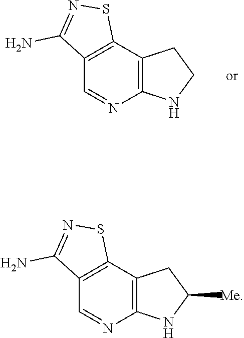

Exemplary compounds of Formula II or Formula III are depicted in Table 3.

TABLE 3
Exemplary Compounds of Formula II and III

| Compound | |
|---|---|
| No. | Structure |
| 1 | 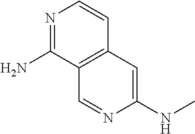 |
| 2 | 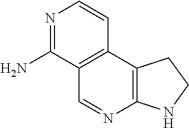 [SMILES: Nc1nccc2c3c(ncc12)NCC3] |
| 3 | 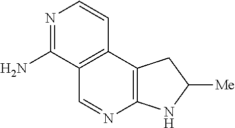 [SMILES: CC1Cc2c(ncc3c(N)nccc23)N1] |
| 4A | 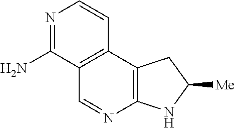 [SMILES: C[C@@H]1Cc2c(ncc3c(N)nccc23)N1] |
| 4B | 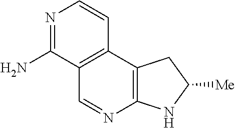 [SMILES: C[C@H]1Cc2c(ncc3c(N)nccc23)N1] |
| 5 | 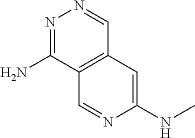 |
| 6 | 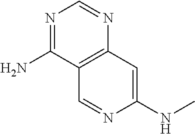 |
| 7 | 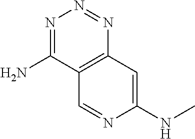 |
| 8 | 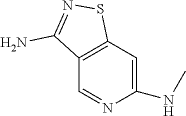 |
| 9 | 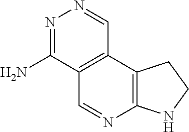 [SMILES: Nc1nncc2c3c(ncc12)NCC3] |
| 10 | 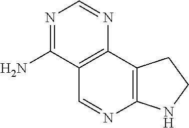 [SMILES: Nc1ncnc2c3c(ncc12)NCC3] |
| 11 | 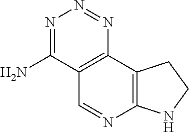 [SMILES: Nc1nnnc2c3c(ncc12)NCC3] |
| 12 | 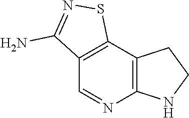 [SMILES: Nc1nsc2c3c(ncc12)NCC3] |
| 13 | 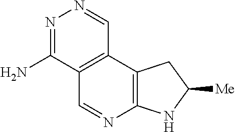 [SMILES: C[C@@H]1Cc2c(ncc3c(N)nncc23)N1] |
| 14 | 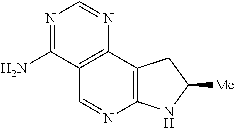 [SMILES: C[C@@H]1Cc2c(ncc3c(N)ncnc23)N1] |
| 15 | 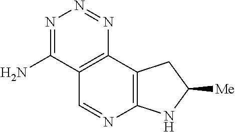 [SMILES: C[C@@H]1Cc2c(ncc3c(N)nnnc23)N1] |
| 16 | 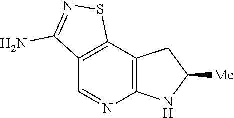 [SMILES: C[C@@H]1Cc2c(ncc3c(N)nsc23)N1] |
| 17 | 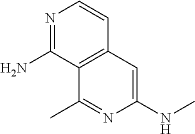 |
| 18 | 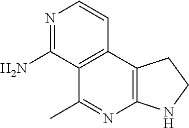 |
| 19 | 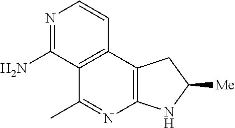 [SMILES: Cc1nc2c(c3ccnc(N)c13)C[C@@H](C)N2] |

### Pharmaceutical Compositions

In certain embodiments, provided herein is a compound described herein together with a pharmaceutically acceptable carrier. In one embodiment, the composition includes a combination of multiple (e.g., two or more) compounds described herein.

In some embodiments, the pharmaceutical composition (i.e., a composition comprising a compound disclosed herein) is delivered locally or systemically. In some embodiments, the pharmaceutical composition may be administered to a tumor present in the subject. In some embodiments, the agent or pharmaceutical composition is administered with an additional cancer therapeutic agent. In some embodiments, the additional cancer therapeutic agent is a chemotherapeutic agent. In some embodiments, the pharmaceutical composition further comprises an additional agent for treatment of cancer. In some embodiments, the additional agent is a tumor vaccine. In certain embodiments, the additional therapeutic agent is a chemotherapeutic agents include alkylating agents such as oxaliplatin, thiotepa, cyclophosphamide (Cytoxan™); alkyl sulfonates such as busulfan, improsulfan and piposulfan; aziridines such as benzodopa, carboquone, meturedopa, and uredepa; emylerumines and memylamelamines including altretamine, triemylenemelamine, triethylenephosphoramide, triethylenethiophosphoramide, and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; antibiotics such as the enediyne antibiotics (e.g., calicheamicin, especially calicheamicin gammall and calicheamicin phili); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (Taxol™, Bristol Meyers Squibb Oncology, Princeton, N.J.) and docetaxel (Taxoteret™, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (Navelbine™); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above. I

Examples of combination therapies with which compounds of the invention may be conjointly administered are included in Table 4.

TABLE 4
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, |
| | Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, |
| | Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, |
| | Vinblastine |
| BCVPP | Carmustine, Cyclophosphamide, |
| | Vinblastine, Procarbazine, Prednisone |
| BEACOPP | Bleomycin, Etoposide, Doxorubicin, |
| | Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, |
| | Mesna |
| BOMP | Bleomycin, Vincristine, Cisplatin, |
| | Mitomycin |
| CA | Cytarabine, Asparaginase |
| CABO | Cisplatin, Methotrexate, Bleomycin, |
| | Vincristine |
| CAF | Cyclophosphamide, Doxorubicin, |
| | Fluorouracil |
| CAL-G | Cyclophosphamide, Daunorubicin, |
| | Vincristine, Prednisone, Asparaginase |
| CAMP | Cyclophosphamide, Doxorubicin, |
| | Methotrexate, Procarbazine |
| CAP | Cyclophosphamide, Doxorubicin, |
| | Cisplatin |
| CaT | Carboplatin, Paclitaxel |
| CAV | Cyclophosphamide, Doxorubicin, |
| | Vincristine |
| CAVE ADD | CAV and Etoposide |
| CA-VP16 | Cyclophosphamide, Doxorubicin, |
| | Etoposide |
| CC | Cyclophosphamide, Carboplatin |
| CDDP/VP-16 | Cisplatin, Etoposide |
| CEF | Cyclophosphamide, Epirubicin, |
| | Fluorouracil |
| CEPP(B) | Cyclophosphamide, Etoposide, |
| | Prednisone, with or without/Bleomycin |
| CEV | Cyclophosphamide, Etoposide, |
| | Vincristine |
| CF | Cisplatin, Fluorouracil or |
| | Carboplatin Fluorouracil |
| CHAP | Cyclophosphamide or Cyclophosphamide, |
| | Altretamine, Doxorubicin, Cisplatin |
| ChlVPP | Chlorambucil, Vinblastine, |
| | Procarbazine, Prednisone |
| CHOP | Cyclophosphamide, Doxorubicin, |
| | Vincristine, Prednisone |
| CHOP-BLEO | Add Bleomycin to CHOP |
| CISCA | Cyclophosphamide, Doxorubicin, |
| | Cisplatin |
| CLD-BOMP | Bleomycin, Cisplatin, Vincristine, |
| | Mitomycin |
| CMF | Methotrexate, Fluorouracil, |
| | Cyclophosphamide |
| CMFP | Cyclophosphamide, Methotrexate, |
| | Fluorouracil, Prednisone |
| CMFVP | Cyclophosphamide, Methotrexate, |
| | Fluorouracil, Vincristine, Prednisone |
| CMV | Cisplatin, Methotrexate, Vinblastine |
| CNF | Cyclophosphamide, Mitoxantrone, |
| | Fluorouracil |
| CNOP | Cyclophosphamide, Mitoxantrone, |
| | Vincristine, Prednisone |
| COB | Cisplatin, Vincristine, Bleomycin |
| CODE | Cisplatin, Vincristine, Doxorubicin, |
| | Etoposide |
| COMLA | Cyclophosphamide, Vincristine, |
| | Methotrexate, Leucovorin, Cytarabine |
| COMP | Cyclophosphamide, Vincristine, |
| | Methotrexate, Prednisone |
| Cooper Regimen | Cyclophosphamide, Methotrexate, |
| | Fluorouracil, Vincristine, Prednisone |
| COP | Cyclophosphamide, Vincristine, |
| | Prednisone |
| COPE | Cyclophosphamide, Vincristine, |
| | Cisplatin, Etoposide |
| COPP | Cyclophosphamide, Vincristine, |
| | Procarbazine, Prednisone |
| CP(Chronic | Chlorambucil, Prednisone |
| lymphocytic leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, |
| | Mesna |
| CVP | Cyclophosphamide, Vincristine, |
| | Prednisome |
| CVPP | Lomustine, Procarbazine, Prednisone |
| CYVADIC | Cyclophosphamide, Vincristine, |
| | Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, |
| | Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, |
| | Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, |
| | Methotrexate, Fluorouracil |
| ICE-T | Ifosfamide, Carboplatin, Etoposide, |
| | Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, |
| | Cyclophosphamide, Prednisone, Melphalan |
| MAC-III | Methotrexate, Leucovorin, Dactinomycin, |
| | Cyclophosphamide |
| MACC | Methotrexate, Doxorubicin, |
| | Cyclophosphamide, Lomustine |
| MACOP-B | Methotrexate, Leucovorin, Doxorubicin, |
| | Cyclophosphamide, Vincristine, |
| | Bleomycin, Prednisone |
| MAID | Mesna, Doxorubicin, Ifosfamide, Dacarbazine |
| m-BACOD | Bleomycin, Doxorubicin, |
| | Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, |
| | Prednisone |
| MOPP/ABV | Mechlorethamine, Vincristine, Procarbazine, |
| | Prednisone, Doxorubicin, Bleomycin, |
| | Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, |
| | Prednisone |
| MTX-CDDPAdr | Methotrexate, Leucovorin, Cisplatin, |
| | Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, |
| | Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, |
| | Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, |
| | Doxorubicin, Cyclophosphamide, Etoposide |
| ProMACE/cytaBOM | Prednisone, Doxorubicin, Cyclophosphamide, |
| | Etoposide, Cytarabine, Bleomycin, |
| | Vincristine, Methotrexate, |
| | Leucovorin, Cotrimoxazole |
| PRoMACE/MOPP | Prednisone, Doxorubicin, Cyclophosphamide, |
| | Etoposide, Mechlorethamine, Vincristine, |
| | Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, |
| | Asparaginase |
| SMF | Streptozocin, Mitomycin, Fluorouracil |
| TAD | Mechlorethamine, Doxorubicin, Vinblastine, |
| | Vincristine, Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, |
| | Cisplatin, Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, |
| | Flouxymesterone |
| VBAP | Vincristine, Carmustine, Doxorubicin, |
| | Prednisone |
| VBCMP | Vincristine, Carmustine, Melphalan, |
| | Cyclophosphamide, Prednisone |
| VC | Vinorelbine, Cisplatin |
| VCAP | Vincristine, Cyclophosphamide, Doxorubicin, |
| | Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, |
| | Prednisone |
| VP | Etoposide, Cisplatin |
| V-TAD | Etoposide, Thioguanine, Daunorubicin, |
| | Cytarabine |
| 5 + 2 | Cytarabine, Daunorubicin, Mitoxantrone |
| 7 + 3 | Cytarabine with/, Daunorubicin or |
| | Idarubicin or Mitoxantrone |
| “8 in 1” | Methylprednisolone, Vincristine, Lomustine, |
| | Procarbazine, Hydroxyurea, Cisplatin, |
| | Cytarabine, Dacarbazine |

In some embodiments, the additional cancer therapeutic agent is an immune checkpoint inhibitor. Immune Checkpoint inhibition broadly refers to inhibiting the checkpoints that cancer cells can produce to prevent or downregulate an immune response. Examples of immune checkpoint proteins are CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof.

Non-limiting examples of immune checkpoint inhibitors are cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, RO7121661, CX-188, atezolizumab (MPDL3280A, RG7446, RO5541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, LY3415244, ipilimumab, tremelimumab and L3D10.

The pharmaceutical compositions and/or agents disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Therapeutic Methods

In some embodiments, the methods described herein may be used to treat any cancerous or pre-cancerous tumor. In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject has cancer. In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor. Actual dosage levels of the active ingredients in the pharmaceutical compositions or agents to be administered may be varied so as to obtain an amount of the active ingredient (e.g., an agent described herein) which is effective to achieve the desired therapeutic response for a particular patient, composition, and mode of administration, without being toxic to the patient.

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day. In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months, for at least 1 year, for at least two years, at least three years, or at least five years.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could prescribe and/or administer doses of the compounds employed in the pharmaceutical composition at levels lower than that required in order to achieve the desired therapeutic effect and gradually increase the dosage until the desired effect is achieved.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing cancer in a subject, the method comprising administering to the subject a compound having the structure of Formula I, Formula II, or Formula III, or a pharmaceutically acceptable salt thereof.

2. A method of decreasing the activity of or lowering the levels of cancer associated fibroblasts in a subject afflicted with cancer, the method comprising administering to the subject a compound having the structure of Formula I, Formula II, or Formula III, or a pharmaceutically acceptable salt thereof.

3. A method of decreasing the activity of or lowering the levels of NNMT in a subject afflicted with cancer, the method comprising administering to the subject a compound having the structure of Formula I, Formula II, or Formula III, or a pharmaceutically acceptable salt thereof.

4. The method of claim 1, wherein the cancer is ovarian cancer or pancreatic cancer.

5. The method of claim 1, wherein the compound is administered with an additional cancer agent.

6. The method of claim 5, wherein the additional cancer agent is chemotherapy.

7. The method of claim 6, wherein the chemotherapy is oxaliplatin.

8. The method of claim 5, wherein the additional cancer agent is an immune checkpoint inhibitor.

9. The method of claim 8, wherein the immune checkpoint inhibitor therapy comprises an inhibitor of an immune checkpoint protein selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof.

10. The method of claim 5, wherein the compound and additional cancer agent exhibit synergy when administered conjointly.
